(19) United States
(12) Patent Application Publication
(10) Pub. No.: US 2016/0368868 A1
(43) Pub. Date: Dec. 22, 2016

(54) N-(1-CYANO-2-HYDROXY-1-METHYL-ETHYL)-4-(TRIFLUOROMETHYLSULFANYL)BENZAMIDE DERIVATIVES FOR USE AS NEMATOCIDAL DRUGS

(71) Applicants: UNIVERSITÄT ZÜRICH Prorektorat MNW, Zürich (CH); UNIVERSITÄT ZÜRICH PROREKTORAT MNW, Zürich (CH)

(72) Inventors: Brendan Robert ANSELL, Clifton Hill, Victoria (AU); Gilles GASSER, Zug (CH); Robin B, GASSER, Werribee, Victoria (AU); Abdul JABBAR, Tarneit, Victoria (AU); Malay PATRA, Zurich (CH); Jeannine HESS, Oberkirch (CH)

(73) Assignees: Universite Zurich, Zurich (CH); The University of Melbourne, Melbourne, Victoria (AU)

(21) Appl. No.: 14/902,007

(22) PCT Filed: Jul. 1, 2014
(86) PCT No.: PCT/EP2014/064006

§ 371 Date: Dec. 30, 2015

(30) Foreign Application Priority Data
Jul. 1, 2013 (EP) 13174553.1

(51) Int. Cl. C07C 323/62; C07C 255/29; C07C 317/44

(52) U.S. Cl. CPC C07C 323/62; C07C 317/44; C07C 255/29

(57) ABSTRACT

The invention relates to compounds characterized by a general formula (1),

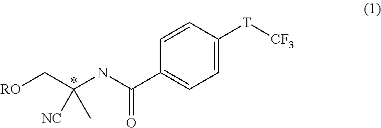

wherein T is S, SO or SO₂, and
R is hydrogen or CO—R′
wherein R′ is unsubstituted or substituted alkyl, alkenyl, alkynyl, aryl, heteroaryl, for use in a method for treatment of infections by helminths.

Parasites cause significant economic losses to agriculture worldwide due to poor productivity, limited growth rates and death. According to some estimates, the financial damage caused by parasites to the livestock industry is in the order of tens of billions of dollars per annum. Decreased productivity influences not only the livestock industry but also substantially affects global food production. Moreover, in spite of the anthelmintic drugs discovered and marketed in the last decades, problems of parasitic worms persist, and multi-drug resistance to most classes of anthelmintics is widespread. The development of new classes of anthelmintics is a major priority. Any anthelmintic developed for parasites of livestock would also have application to parasites of humans and other animals, including companion animals, such as dogs, cats and equids. One sixth of the human population on earth is affected chronically by at least one parasitic helminth, and the socioeconomic burden (in DALYs) is greater than that of cancer and diabetes. Some helminths, such as Schistosoma haematobium, Opisthorchis viverrini and Clonorchis sinensis induce malignant cancers in humans.

Recently, a new class of synthetic anthelmintics, called Amino-Acetonitrile Derivatives (AADs, see WO2005044784A1), has been developed commercially under the trade name Zolvix for the treatment of infected sheep.

### Monepantel (AAD 1566)

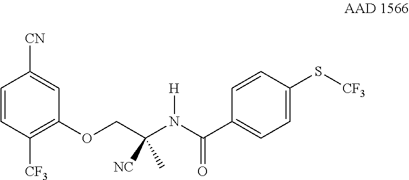 [SMILES: C[C@](C#N)(COc1cc(C#N)ccc1C(F)(F)F)NC(=O)c1ccc(SC(F)(F)F)cc1]

The precise mode of its action is not yet known, although an interaction of AADs with a specific acetylcholine receptor (nAChR) subunit has been proposed. This target is only present in nematodes but not in mammals, making it relevant for the development of a new class of antihelmintic drugs. A mutant of Haemonchus contortus with a reduced sensitivity to monepantel was recently identified using a novel in vitro selection procedure. This result indicates that resistance to monepantel might develop in the future.

In light of the above referenced state of the art, the objective of the present invention is to provide novel compounds to control parasitic nematodes of human beings and livestock. This objective is attained by the subject matter of the independent claims.

### SUMMARY OF THE INVENTION

The present invention was made during the course of an investigation into the potential of novel AAD derivatives that are smaller structural analogues of monepantel. In order to evaluate the potential of these compounds as nematocidal drug candidates, a screen was undertaken on Haemonchus contortus (H. contortus). This nematode is ideal, because it is relatively closely related to arguably the most economically important species of parasitic nematodes (order Strongylida; clade V) of livestock.

According to a first aspect of the invention, a compound is provided for use in a method for treatment of helminth infections. This compound is characterized by a general formula (A),

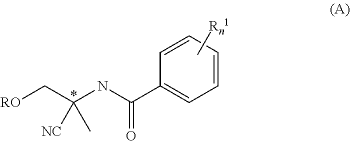

wherein
n of R¹ₙ is 0, 1, 2, 3, 4 or 5, and
each R¹ independently from any other R¹ is —C(═O)OR^{2a}, —C(═O)NR^{2a}₂, —C(═O)SR^{2a}, —C(═S)OR^{2a}, —C(NH)NR^{2a}₂, CN₄H₂, —NR^{2a}₂, —C(═O)R^{2a}, —C(═S)R^{2a}, —OR^{2a}, —SR^{2a}, —CF₃, —OCF₃, —SCF₃, —SOCF₃, —SO₂CF₃, —CN, —NO₂, —F, —Cl, —Br or —I,
with each R^{2a} independently from any other R^{2a} being a hydrogen or C₁-C₄ alkyl, and
R is hydrogen or CO—R′
   wherein R′ is
   an unsubstituted or substituted C₁-C₁₀ alkyl, an unsubstituted or substituted C₂-C₁₀ alkenyl, an unsubstituted or substituted C₂-C₁₀ alkynyl, an unsubstituted or substituted C₃-C₈ cycloalkyl, an unsubstituted or substituted C₁-C₁₀ alkoxy, an unsubstituted or substituted C₃-C₈ cycloalkoxy,
   an unsubstituted or substituted C₆-C₁₄ aryl,
   an unsubstituted or substituted 5- to 10-membered heteroaryl, wherein 1 to 4 ring atoms are independently selected from nitrogen, oxygen or sulfur,
   an unsubstituted or substituted 5- to 10-membered heteroalicyclic ring, wherein 1 to 3 ring atoms are independently nitrogen, oxygen or sulfur,
   —OR², —C(O)R², —C(O)OR², —C(O)NR²R³, —NR²R³, —S(O)₂R², —S(O)₂OR², and —S(O)₂NR²R³,
   wherein
      R² and R³ are independently selected from the group consisting of hydrogen, unsubstituted C₁-C₄ alkyl, and C₁-C₄ alkyl substituted with C₁-C₄ alkoxy.

According to a sub aspect of the first aspect a compound is provided for use in a method for treatment of helminth infections. This compound is characterized by a general formula (1),

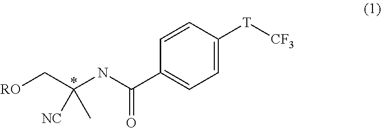

wherein T is S, SO or SO₂, and
R is hydrogen or CO—R′
   wherein R′ is
   an unsubstituted or substituted C₁-C₁₀ alkyl, an unsubstituted or substituted C₁-C₁₀ alkenyl, an unsubstituted or substituted C₁-C₁₀ alkynyl, an unsubstituted or substituted C₃-C₈ cycloalkyl, an unsubstituted or substituted C₁-C₁₀ alkoxy, an unsubstituted or substituted C₃-C₈ cycloalkoxy,
   an unsubstituted or substituted C₆-C₁₄ aryl,
   an unsubstituted or substituted 5- to 10-membered heteroaryl, wherein 1 to 4 ring atoms are independently selected from nitrogen, oxygen or sulfur,
   an unsubstituted or substituted 5- to 10-membered heteroalicyclic ring, wherein 1 to 3 ring atoms are independently nitrogen, oxygen or sulfur,
   —OR², —C(O)R², —C(O)OR², —C(O)NR²R³, —NR²R³, —S(O)₂R², —S(O)₂OR², and —S(O)₂NR²R³,
      wherein
      R² and R³ are independently selected from the group consisting of hydrogen, unsubstituted C₁-C₄ alkyl, and C₁-C₄ alkyl substituted with C₁-C₄ alkoxy.

Helminths, in the context of the present invention, are parasitic worms, particularly tapeworms (cestodes), flukes (trematodes) and roundworms (nematodes). Particular exemplary indications in humans include, but are not limited to, tapeworm infection, fascioliasis, schistosomiasis, ascariasis, dracunculiasis, elephantiasis, enterobiasis, filariasis, hookworm infection/disease, onchocerciasis, trichinellosis and trichuriasis.

A C₁-C₁₀ alkyl in the context of the present invention signifies a saturated linear or branched hydrocarbon having 1, 2, 3, 4, 5, 6, 7, 8, 9 or 10 carbon atoms, wherein one CH₂ moiety may be exchanged for oxygen (ether bridge). Likewise, a C₁-C₄ alkyl signifies a saturated linear or branched hydrocarbon having 1, 2, 3 or 4 carbon atoms. Non-limiting examples for a C₁-C₄ alkyl are methyl, ethyl, propyl, n-butyl, 2-methylpropyl or tert-butyl. Non-limiting examples for a C₅ alkyl include n-pentyl, 2-methylbutyl, 3-methylbutyl, 1,1-dimethylpropyl, 1,2-dimethylpropyl, 1,2-dimethylpropyl and pent-4-inyl.

A C₂-C₁₀ alkenyl in the context of the present invention signifies a linear or branched hydrocarbon having 2, 3, 4, 5, 6, 7, 8, 9 or 10 carbon atoms, wherein one or more carbon-carbon bonds is unsaturated and one CH₂ moiety may be exchanged for oxygen (ether bridge). Non-limiting examples for a C₂-C₅ alkenyl are ethenyl, prop-2-enyl, but-3-enyl, 3-methylbut-2-enyl, 2-methylbut-3-enyl, and 3-methylbut-3-enyl.

A C₂-C₁₀ alkynyl in the context of the present invention signifies a linear or branched hydrocarbon having 2, 3, 4, 5, 6, 7, 8, 9 or 10 carbon atoms, wherein one or more carbon-carbon bonds is doubly unsaturated and one CH₂ moiety may be exchanged for oxygen (ether bridge). Alkynyl moieties may comprise both doubly unsaturated and mono-unsaturated carbon-carbon bonds.

The term aryl in the context of the present invention signifies a cyclic aromatic hydrocarbon. A heteroaryl in the context of the present invention refers to an aryl that comprises one or several nitrogen, oxygen and/or sulphur atoms. An aryl or heteroaryl may be substituted by one or more functional groups selected from COOR⁵, CONR⁵₂, C(NH)NR⁵₂, CN₄H₂, NR⁵₂, COR⁵, OR⁵, CF₃, OCF₃, SCF₃, SOCF₃, SO₂CF₃, CN, NO₂, F, Cl or Br, with each R⁵ independently from any other being hydrogen or a C₁-C₄ alkyl. Such functional group may enhance the solubility in an aqueous medium of the compound of the invention.

A heteroalicyclic ring in the context of the present invention signifies a cyclic saturated or partially unsaturated, but not aromatic, hydrocarbon. Non-limiting examples of heteroalicyclic rings are thiolane, imidazolidine, pyrazolidine, oxazolidine, isoxazolidine, thiazolidine, dioxolane, dithiolane, piperidine, tetrahydrofurane, piperazine, morpholine, thiomorpholine, dioxane, dithiane, azepane, oxepane and thiepane.

The following embodiments relate to compounds according to formula 1 or formula A.

In some embodiments, R′ is an unsubstituted C₁-C₅ alkyl.

In some embodiments, R′ is a C₂-C₅ alkenyl. In some embodiments, R′ is a C₂-C₅ alkynyl. In some embodiments, wherein R′ is a C₁-C₁₀ alkyl, a C₁-C₅ alkyl, a C₂-C₁₀ or C₂-C₅ alkenyl, or a C₂-C₁₀ or a C₂-C₅ alkynyl, R′ is substituted by one, two or three functional group(s) selected from C(NH)NR⁵₂, CN₄H₂, NR⁵₂, COOR⁵, CONR⁵₂, COR⁵, CF₃, OCF₃, SCF₃, SOCF₃, SO₂CF₃, OR⁵, CN, NO₂, F, Cl, and Br, wherein each R⁵ independently from any other is hydrogen or a C₁-C₄ alkyl. In some embodiments, R⁵ is an unsubstituted C₁-C₄ alkyl. In some embodiments, R′ is a mono-substituted C₁-C₁₀ alkyl, a C₂-C₁₀ or C₂-C₅ alkenyl, or a C₂-C₁₀ or a C₂-C₅ alkynyl, having one functional group (substitution) in co-position (terminal position on the alkyl/alkenyl/alkynyl chain), selected from C(NH)NR⁵₂, CN₄H₂, NR⁵₂, COOR⁵, CONR⁵₂, COR⁵, CF₃, OCF₃, SCF₃, SOCF₃, SO₂CF₃, OR⁵, CN, NO₂, F, Cl, and Br, wherein each R⁵ independently from any other is hydrogen or an unsubstituted C₁-C₄ alkyl.

Similarly to the alkyl moieties discussed above, the heteroalicyclic ring may be substituted by one, two or three functional group(s) selected from C(NH)NR⁵₂, CN₄H₂, NR⁵₂, COOR⁵, CONR⁵₂, COR⁵, CF₃, OCF₃, SCF₃, SOCF₃, SO₂CF₃, OR⁵, CN, NO₂, F, Cl, and Br, wherein each R⁵ independently from any other is hydrogen or a C₁-C₄ alkyl.

A C₁-C₄ alkoxy moiety in the context of the present invention signifies a C₁-C₄ alkyl moiety according to the above definition, linked to the respective moiety by an oxygen (ether).

In some embodiments, n of R¹ₙ is 1 or 2, and each R¹ independently from any other R¹ is —C(═O)OR^{2a}, —C(═O)NR^{2a}₂, —C(═O)SR^{2a}, —C(═S)OR^{2a}, —C(NH)NR^{2a}₂, CN₄H₂, —NR^{2a}₂, —C(═O)R^{2a}, —C(═S)R^{2a}, —OR^{2a}, —SR^{2a}, —CF₃, —OCF₃, —SCF₃, —SOCF₃, —SO₂CF₃, —CN, —NO₂, —F, —Cl, —Br or —I, with each R^{2a} independently from any other R^{2a} being hydrogen, CH₃, C₂H₅, C₃H₇ or C₄H₉, in particular with each R^{2a} being hydrogen.

In some embodiments, n of R¹ₙ is 1 or 2 and each R¹ independently from any other R¹ is —CF₃, —OCF₃, —SCF₃, —SOCF₃, —SO₂CF₃, —F, —Cl, —Br or —I. In some embodiments, n of R¹ₙ is 1 or 2 and each R¹ independently from any other R¹ is —CN, —CF₃, —SCF₃, —SOCF₃ or —SO₂CF₃. In some embodiments, n of R¹ₙ is 1 or 2 and each R¹ independently from any other R¹ is —F, —Cl, —Br or —I.

In some embodiments, n of R¹ₙ is 2 and each R¹ independently from any other R¹ is —CN, —CF₃, —OCF₃, —F, —Cl, —Br or —I. In some embodiments, n of R¹ₙ is 2 and each R¹ independently from any other R¹ is —CN or —CF₃.

In some embodiments, n of R¹ₙ is 2 and one of the two R¹ is in ortho and the other R¹ is in meta position to the attachment position of the benzene moiety. In some embodiments, n of R¹ₙ is 2, each R¹ independently from any other R¹ is —CN, —CF₃, —OCF₃, —SCF₃, —SOCF₃, —SO₂CF₃, —F, —Cl, —Br or —I, in particular each R¹ independently from any other R¹ is —CN, —CF₃, —OCF₃, —F, —Cl or —Br, and one of the two R¹ is in ortho and the other R¹ is in meta position to the attachment position of the benzene moiety.

In some embodiments, n of R¹ₙ is 2, each R¹ independently from any other R¹ is —CN or —CF₃ and one of the two R¹ is in ortho and the other R¹ is in meta position to the attachment position of the benzene moiety. In some embodiments, n of R¹ₙ is 2 and one of the two R¹ is —CF₃ in ortho and the other R¹ is —CN in meta position to the attachment position of the benzene moiety.

In some embodiments, n of R¹ₙ is 1 and R¹ is —CN, —CF₃, —OCF₃, —SCF₃, —SOCF₃, —SO₂CF₃, —F, —Cl, —Br or —I. In some embodiments, n of R¹ₙ is 1 and R¹ is —SCF₃, —SOCF₃ or —SO₂CF₃, in particular R¹ is —SCF₃.

In some embodiments, n of R¹ₙ is 1 and R¹ is in para position to the attachment position of the benzene moiety. In some embodiments, n of R¹ₙ is 1, R¹ is —CN, —CF₃, —OCF₃, —SCF₃, —SOCF₃, —SO₂CF₃, —F, —Cl, —Br or —I, in particular R¹ is —SCF₃, —SOCF₃, —SO₂CF₃, —F, —Cl or —Br, and R¹ is in para position to the attachment position of the benzene moiety.

In some embodiments, n of R¹ₙ is 1 and R¹ is —SCF₃, —SOCF₃, —SO₂CF₃ and R¹ is in para position to the attachment position of the benzene moiety. In some embodiments, n of R¹ₙ is 1, R¹ is —SCF₃ and R¹ is in para position to the attachment position of the benzene moiety.

Pharmaceutically acceptable salts of the compounds provided herein are deemed to be encompassed by the scope of the present invention.

In some embodiments, R′ is an aryl or a heteroaryl selected from the group comprised of:

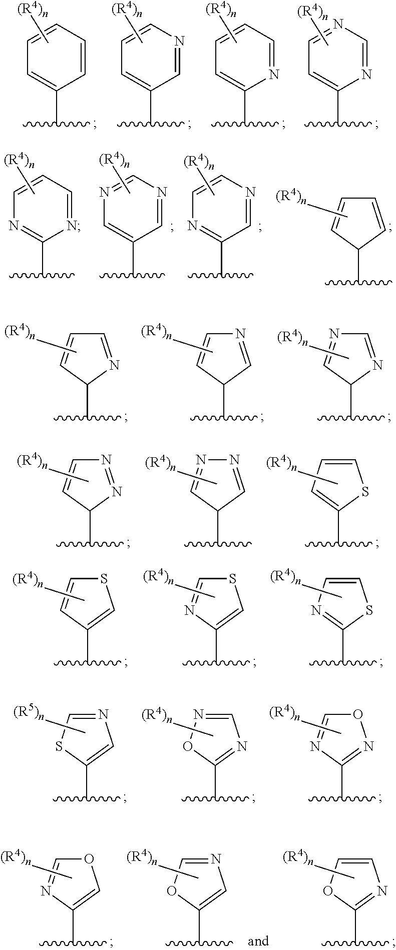

wherein
n is 0, 1, 2, 3 or 4, and
each R⁴ independently from any other is COOR⁵, CONR⁵₂, C(NH)NR⁵₂, CN₄H₂, NR⁵₂, COR⁵, OR⁵, CF₃, OCF₃, SCF₃, SOCF₃, SO₂CF₃, CN, NO₂, F, Cl or Br,
with each R⁵ independently from any other being hydrogen or a C₁-C₄ alkyl.

In some embodiments, R′ is substituted by one or two R⁴ groups (n is 1 or 2).

In some embodiments, R′ is phenyl, 2-pyridyl, 3-pyridyl, 4-pyridyl, 2-pyrimidyl, 4-pyrimidyl or 5-pyrimidyl. In some embodiments, R′ is a six-membered ring substituted by one or two R⁴ substituents in para and/or ortho position to the attachment position of R⁴.

In some embodiments, R⁴ is a CF₃, OCF₃, SCF₃, SOCF₃, or SO₂CF₃ group. In one embodiment, R⁴ is SCF₃ and T is S.

In some embodiments, each R⁵ independently from any other is hydrogen, CH₃, C₂H₅, C₃H₇ or C₄H₉.

In some embodiments, R′ is selected from the group comprised of:

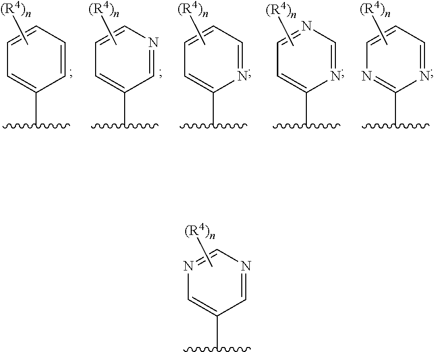

wherein n is 1 and R⁴ is CF₃, OCF₃, SCF₃, SOCF₃, or SO₂CF₃.

In some embodiments, R′ is selected from
4-(trifluoromethylsulfanyl)phenyl,
4-(trifluoromethylsulfinyl)phenyl, or
4-(trifluoromethylsulfonyl)phenyl.

Particular embodiments of the compound of the invention for use in a method for treatment of infection by helminths are selected from
a. N-(1-cyano-2-hydroxy-1-methyl-ethyl)-4-(trifluoromethylsulfanyl)benzamide (ahp-OH);

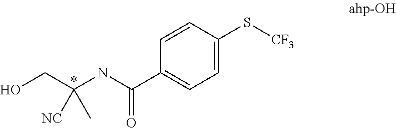 [SMILES: CC(C#N)(CO)NC(=O)c1ccc(SC(F)(F)F)cc1]

b. N-(1-cyano-2-hydroxy-1-methyl-ethyl)-4-(trifluoromethylsulfinyl)benzamide

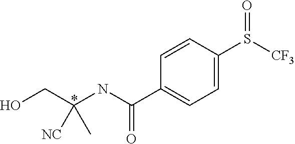 [SMILES: CC(C#N)(CO)NC(=O)c1ccc(S(=O)C(F)(F)F)cc1]

c. N-(1-cyano-2-hydroxy-1-methyl-ethyl)-4-(trifluoromethylsulfonyl)benzamide

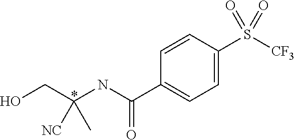 [SMILES: CC(C#N)(CO)NC(=O)c1ccc(S(=O)(=O)C(F)(F)F)cc1]

d. [2-cyano-2-[[4-(trifluoromethylsulfanyl)benzoyl]amino]propyl] 4-(trifluoromethylsulfanyl)benzoate

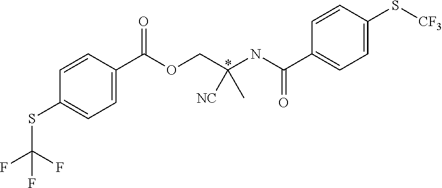 [SMILES: CC(C#N)(COC(=O)c1ccc(SC(F)(F)F)cc1)NC(=O)c1ccc(SC(F)(F)F)cc1]

e. [2-cyano-2-[[4-(trifluoromethylsulfanyl)benzoyl]amino]propyl] acetate

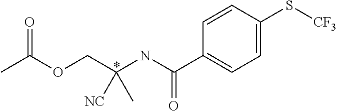 [SMILES: CC(=O)OCC(C)(C#N)NC(=O)c1ccc(SC(F)(F)F)cc1]

f. N-(2-cyano-1-hydroxypropan-2-yl)-4-(trifluoromethoxy)benzamide

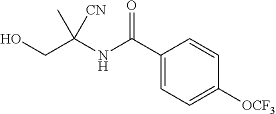 [SMILES: CC(C#N)(CO)NC(=O)c1ccc(OC(F)(F)F)cc1]

g. N-(2-cyano-1-hydroxypropan-2-yl)-4-(trifluoromethyl)benzamide

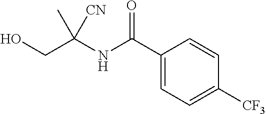 [SMILES: CC(C#N)(CO)NC(=O)c1ccc(C(F)(F)F)cc1]

h. N-(2-cyano-1-hydroxypropan-2-yl)-4-(methylthio)benzamide

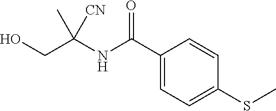 [SMILES: CSc1ccc(C(=O)NC(C)(C#N)CO)cc1]

i. N-(2-cyano-1-hydroxypropan-2-yl)-4-fluorobenzamide

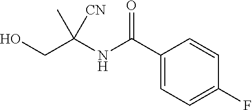 [SMILES: CC(C#N)(CO)NC(=O)c1ccc(F)cc1]

j. 4-chloro-N-(2-cyano-1-hydroxypropan-2-yl)benzamide

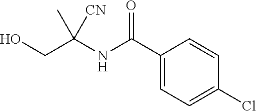 [SMILES: CC(C#N)(CO)NC(=O)c1ccc(Cl)cc1]

k. 4-bromo-N-(2-cyano-1-hydroxypropan-2-yl)benzamide

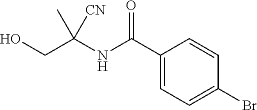 [SMILES: CC(C#N)(CO)NC(=O)c1ccc(Br)cc1]

l. N-(2-cyano-1-hydroxypropan-2-yl)-4-iodobenzamide

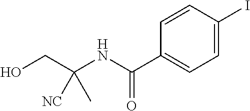 [SMILES: CC(C#N)(CO)NC(=O)c1ccc(I)cc1]

m. 2-cyano-2-(4-((trifluoromethyl)thio)benzamido)propyl benzoate

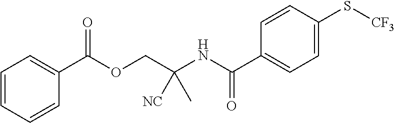 [SMILES: CC(C#N)(COC(=O)c1ccccc1)NC(=O)c1ccc(SC(F)(F)F)cc1]

n. 2-cyano-2-(4-((trifluoromethyl)thio)benzamido)propyl propiolate

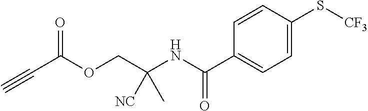 [SMILES: C#CC(=O)OCC(C)(C#N)NC(=O)c1ccc(SC(F)(F)F)cc1]

According to a second aspect of the invention, a compound is provided characterized by a general formula (B),

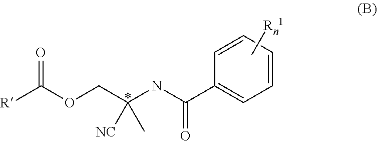

wherein
n of R¹ₙ is 0, 1, 2, 3, 4 or 5, and
each R¹ independently from any other R¹ is —C(═O)OR^{2a}, —C(═O)NR^{2a}₂, —C(═O)SR^{2a}, —C(═S)OR^{2a}, —C(NH)NR^{2a}₂, CN₄H₂, —NR^{2a}₂, —C(═O)R^{2a}, —C(═S)R^{2a}, —OR^{2a}, —SR^{2a}, —CF₃, —OCF₃, —SCF₃, —SOCF₃, —SO₂CF₃, —CN, —NO₂, —F, —Cl, —Br or —I,
with each R^{2a} independently from any other R^{2a} being a hydrogen or C₁-C₄ alkyl, and
R′ is
   an unsubstituted or substituted C₁-C₁₀ alkyl, an unsubstituted or substituted C₂-C₁₀ alkenyl, an unsubstituted or substituted C₂-C₁₀ alkynyl, an unsubstituted or substituted C₃-C₈ cycloalkyl, an unsubstituted or substituted C₁-C₁₀ alkoxy, an unsubstituted or substituted C₃-C₈ cycloalkoxy,
   an unsubstituted or substituted C₆-C₁₄ aryl,
   an unsubstituted or substituted 5- to 10-membered heteroaryl, wherein 1 to 4 ring atoms are independently selected from nitrogen, oxygen or sulfur,
   an unsubstituted or substituted 5- to 10-membered heteroalicyclic ring, wherein 1 to 3 ring atoms are independently nitrogen, oxygen or sulfur,
   —OR², —C(O)R¹, —C(O)OR², —C(O)NR²R³, —NR²R³, —S(O)₂R², —S(O)₂OR², and —S(O)₂NR²R³,
      wherein
      R² and R³ are independently selected from the group consisting of hydrogen, unsubstituted C₁-C₄ alkyl, and C₁-C₄ alkyl substituted with C₁-C₄ alkoxy.

According to a sub aspect of the second aspect of the invention, a compound is provided characterized by a general formula (2),

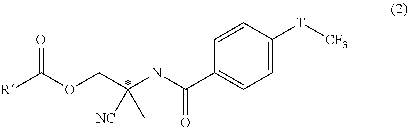

wherein T is S, SO or SO₂, and
R′ is
   an unsubstituted or substituted C₁-C₁₀ alkyl, an unsubstituted or substituted C₁-C₁₀ alkenyl, an unsubstituted or substituted C₁-C₁₀ alkynyl, an unsubstituted or substituted C₃-C₈ cycloalkyl, an unsubstituted or substituted C₁-C₁₀ alkoxy, an unsubstituted or substituted C₃-C₈ cycloalkoxy,
   an unsubstituted or substituted C₆-C₁₄ aryl,
   an unsubstituted or substituted 5- to 10-membered heteroaryl, wherein 1 to 4 ring atoms are independently selected from nitrogen, oxygen or sulfur,
   an unsubstituted or substituted 5- to 10-membered heteroalicyclic ring, wherein 1 to 3 ring atoms are independently nitrogen, oxygen or sulfur,
   —OR², —C(O)R², —C(O)OR², —C(O)NR²R³, —NR²R³, —S(O)₂R², —S(O)₂OR², and —S(O)₂NR²R³,
      wherein
      R² and R³ are independently selected from the group consisting of hydrogen, unsubstituted C₁-C₄ alkyl, and C₁-C₄ alkyl substituted with C₁-C₄ alkoxy.

The following embodiments relate to a compound according to formula 2 or B.

In some embodiments, R′ is an unsubstituted C₁-C₅ alkyl.

In some embodiments, R′ is a C₂-C₅ alkenyl. In some embodiments, R′ is a C₂-C₅ alkynyl.

In some embodiments, wherein R′ is a C₁-C₁₀ alkyl, a C₁-C₅ alkyl, a C₂-C₁₀ or C₂-C₅ alkenyl, or a C₂-C₁₀ or a C₂-C₅ alkynyl, R′ is substituted by one, two or three functional group(s) selected from C(NH)NR⁵₂, CN₄H₂, NR⁵₂, COOR⁵, CONR⁵₂, COR⁵, CF₃, OCF₃, SCF₃, SOCF₃, SO₂CF₃, OR⁵, CN, NO₂, F, Cl, and Br, wherein each R⁵ independently from any other is hydrogen or a C₁-C₄ alkyl. In some embodiments, R⁵ is an unsubstituted C₁-C₄ alkyl. In some embodiments, R′ is a mono-substituted C₁-C₁₀ alkyl, a C₂-C₁₀ or C₂-C₅ alkenyl, or a C₂-C₁₀ or a C₂-C₅ alkynyl, having one functional group (substitution) in ω-position (terminal position on the alkyl/alkenyl/alkynyl chain), selected from C(NH)NR⁵₂, CN₄H₂, NR⁵₂, COOR⁵, CONR⁵₂, COR⁵, CF₃, OCF₃, SCF₃, SOCF₃, SO₂CF₃, OR⁵, CN, NO₂, F, Cl, and Br, wherein each R⁵ independently from any other is hydrogen or an unsubstituted C₁-C₄ alkyl.

In some embodiments, n of R¹ₙ is 1 or 2, and each R¹ independently from any other R¹ is —C(═O)OR^{2a}, —C(═O)NR^{2a}₂, —C(═O)SR^{2a}, —C(═S)OR^{2a}, —C(NH)NR^{2a}₂, CN₄H₂, —NR^{2a}₂, —C(═O)R^{2a}, —C(═S)R^{2a}, —OR^{2a}, —SR^{2a}, —CF₃, —OCF₃, —SCF₃, —SOCF₃, —SO₂CF₃, —CN, —NO₂, —F, —Cl, —Br or —I, with each R^{2a} independently from any other R^{2a} being hydrogen, CH₃, C₂H₅, C₃H₇ or C₄H₉, in particular with each R² being hydrogen.

In some embodiments, n of R¹ₙ is 1 or 2 and each R¹ independently from any other R¹ is —CN, —CF₃, —OCF₃, —SCF₃, —SOCF₃, —SO₂CF₃, —F, —Cl, —Br or —I. In some embodiments, n of R¹ₙ is 1 or 2 and each R¹ independently from any other R¹ is —CN, —CF₃, —SCF₃, —SOCF₃ or —SO₂CF₃. In some embodiments, n of R¹ₙ is 1 or 2 and each R¹ independently from any other R¹ is —F, —Cl, —Br or —I.

In some embodiments, n of R¹ₙ is 2 and each R¹ independently from any other R¹ is —CN, —CF₃, —OCF₃, —F, —Cl, —Br or —I. In some embodiments, n of R¹ₙ is 2 and each R¹ independently from any other R¹ is —CN or —CF₃.

In some embodiments, n of R¹ₙ is 2 and one of the two R¹ is in ortho and the other R¹ is in meta position to the attachment position of the benzene moiety. In some embodiments, n of R¹ is 2, each R¹ independently from any other R¹ is —CN, —CF₃, —OCF₃, —SCF₃, —SOCF₃, —SO₂CF₃—F, —Cl, —Br or —I, in particular each R¹ independently from any other R¹ is —CN, —CF₃, —OCF₃, —F, —Cl or —Br, and one of the two R¹ is in ortho and the other R¹ is in meta position to the attachment position of the benzene moiety.

In some embodiments, n of R¹ₙ is 2, each R¹ independently from any other R¹ is —CN or —CF₃ and one of the two R¹ is in ortho and the other R¹ is in meta position to the attachment position of the benzene moiety. In some embodiments, n of R¹ₙ is 2 and one of the two R¹ is —CF₃ in ortho and the other R¹ is —CN in meta position to the attachment position of the benzene moiety.

In some embodiments, n of R¹ₙ is 1 and R¹ is —CN, —CF₃, —OCF₃, —SCF₃, —SOCF₃, —SO₂CF₃, —F, —Cl, —Br or —I. In some embodiments, n of R¹ₙ is 1 and R¹ is —SCF₃, —SOCF₃ or —SO₂CF₃, in particular R¹ is —SCF₃.

In some embodiments, n of R¹ₙ is 1 and R¹ is in para position to the attachment position of the benzene moiety. In some embodiments, n of R¹ₙ is 1, R¹ is —CN, —CF₃, —OCF₃, —SCF₃, —SOCF₃, —SO₂CF₃, —F, —Cl, —Br or —I, in particular R′ is —SCF₃, —SOCF₃, —SO₂CF₃, —F, —Cl or —Br, and R¹ is in para position to the attachment position of the benzene moiety.

In some embodiments, n of R¹ₙ is 1 and R¹ is —SCF₃, —SOCF₃, —SO₂CF₃ and R¹ is in para position to the attachment position of the benzene moiety. In some embodiments, n of R′ₙ is 1, R¹ is —SCF₃ and R¹ is in para position to the attachment position of the benzene moiety.

In some embodiments, R′ is an aryl or a heteroaryl selected from the group comprised of:

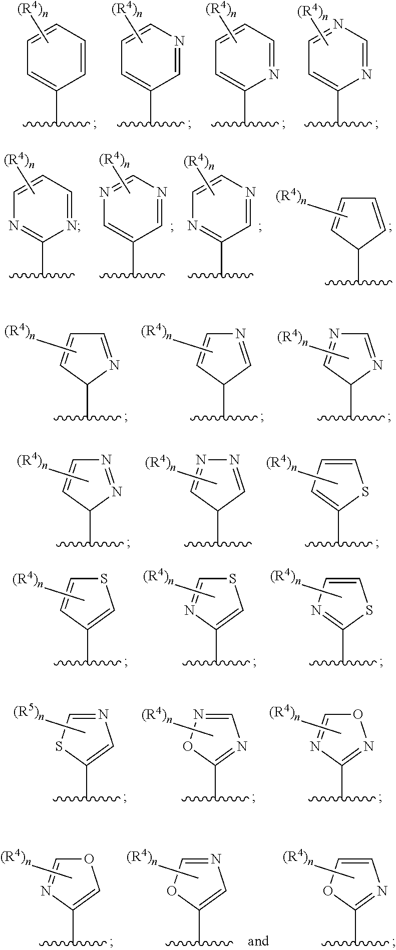

wherein
n is 0, 1, 2, 3 or 4, and
each R⁴ independently from any other is COOR⁵, CONR⁵₂, C(NH)NR⁵₂, CN₄H₂, NR⁵₂, COR⁵, OR⁵, CF₃, OCF₃, SCF₃, SOCF₃, SO₂CF₃, CN, NO₂, F, Cl or Br,
with each R⁵ independently from any other being hydrogen or a C₁-C₄ alkyl.

In some embodiments, R′ is substituted by one or two R⁴ groups (n is 1 or 2).

In some embodiments, R′ is phenyl, 2-pyridyl, 3-pyridyl, 4-pyridyl, 2-pyrimidyl, 4-pyrimidyl or 5-pyrimidyl. In some embodiments, R′ is a six-membered ring substituted by one or two R⁴ substituents in para and/or ortho position to the attachment position of R⁴.

In some embodiments, R⁴ is aCF₃, OCF₃, SCF₃, SOCF₃, or SO₂CF₃ group. In one embodiment, R⁴ is SCF₃ and T is S.

In some embodiments, each R⁵ independently from any other is hydrogen, CH₃, C₂H₅, C₃H₇ or C₄H₉.

In some embodiments, R′ is selected from the group comprised of:

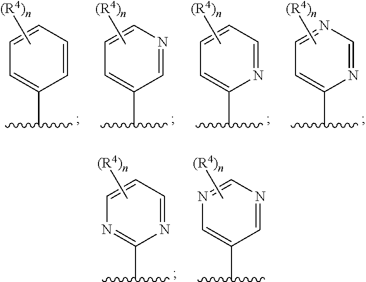

wherein n is 1 and R⁴ is CF₃, OCF₃, SCF₃, SOCF₃, or SO₂CF₃.

In some embodiments, R′ is selected from
4-(trifluoromethylsulfanyl)phenyl,
4-(trifluoromethylsulfinyl)phenyl, or
4-(trifluoromethylsulfonyl)phenyl.

Particular embodiments of this second aspect of the invention are:
a. [2-cyano-2-[[4-(trifluoromethylsulfanyl)benzoyl]amino]propyl] 4-(trifluoromethylsulfanyl)benzoate

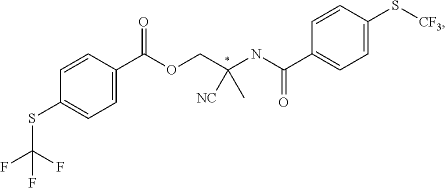 [SMILES: CC(C#N)(COC(=O)c1ccc(SC(F)(F)F)cc1)NC(=O)c1ccc(SC(F)(F)F)cc1]

particularly the S-enantiomer:

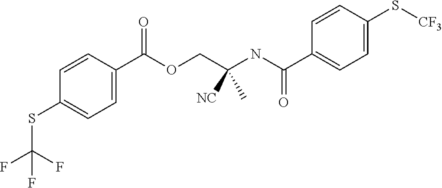 [SMILES: C[C@](C#N)(COC(=O)c1ccc(SC(F)(F)F)cc1)NC(=O)c1ccc(SC(F)(F)F)cc1]

b. [2-cyano-2-[[4-(trifluoromethylsulfanyl)benzoyl]amino]propyl] acetate

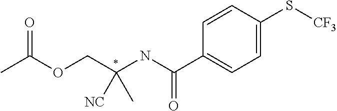 [SMILES: CC(=O)OCC(C)(C#N)NC(=O)c1ccc(SC(F)(F)F)cc1]

particularly the S-enantiomer:

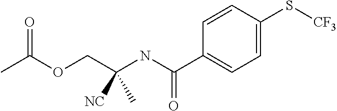 [SMILES: CC(=O)OC[C@](C)(C#N)NC(=O)c1ccc(SC(F)(F)F)cc1]

c. 2-cyano-2-(4-((trifluoromethyl)thio)benzamido)propyl benzoate

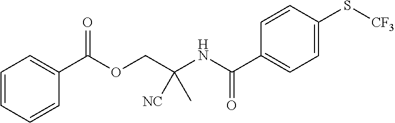 [SMILES: CC(C#N)(COC(=O)c1ccccc1)NC(=O)c1ccc(SC(F)(F)F)cc1]

d. 2-cyano-2-(4-((trifluoromethyl)thio)benzamido)propyl propiolate

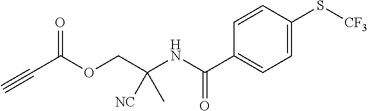 [SMILES: C#CC(=O)OCC(C)(C#N)NC(=O)c1ccc(SC(F)(F)F)cc1]

e. 2-cyano-2-[[4-(trifluoromethylsulfanyl)benzoyl]amino]propyl] 4-(trifluoromethylsulfanyl)benzoate

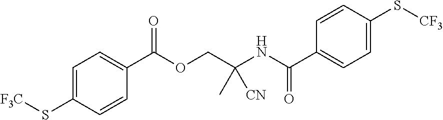 [SMILES: CC(C#N)(COC(=O)c1ccc(SC(F)(F)F)cc1)NC(=O)c1ccc(SC(F)(F)F)cc1]

According to a third aspect of the invention, the compounds defined as the second aspect of the invention are provided for use in a method for treatment or prevention of disease.

In some embodiments, any compound provided herein is provided as an essentially pure stereoisomer. The stereocenter of the marked with an asterisk in formula (1) and (2) (or A and B) is the C1 carbon atom of the ethyl moiety. In some embodiments, essentially pure preparations of a stereoisomer are provided where this atom is in the S configuration.

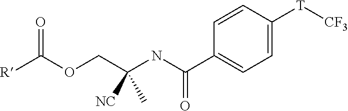

Pharmaceutically acceptable salts of the compounds provided herein are deemed to be encompassed by the scope of the present invention.

According to one aspect of the invention, a pharmaceutical composition for preventing or treating helminth infection, particularly infection by tapeworms (cestodes), flukes (trematodes) and roundworms (nematodes), tapeworm infection, schistosomiasis, ascariasis, dracunculiasis, elephantiasis, enterobiasis, filariasis, hookworm infection, onchocerciasis, trichinosis and/or trichuriasis is provided, comprising a compound according to the above aspect or embodiments of the invention. Pharmaceutical compositions for enteral administration, such as nasal, buccal, rectal or, especially, oral administration, and for parenteral administration, such as dermal (spot-on), intradermal, subcutaneous, intravenous, intrahepatic or intramuscular administration, may be used. The pharmaceutical compositions comprise approximately 1% to approximately 95% active ingredient, preferably from approximately 20% to approximately 90% active ingredient.

According to one aspect of the invention, a dosage form for preventing or treating helminth infection, particularly infection by particularly tapeworms (cestodes), flukes (trematodes) and roundworms (nematodes), tapeworm infection, schistosomiasis, ascariasis, dracunculiasis, elephantiasis, enterobiasis, filariasis, hookworm infection, onchocerciasis, trichinosis and/or trichuriasis is provided, comprising a compound according to the above aspect or embodiments of the invention. Dosage forms may be for administration via various routes, including nasal, buccal, rectal, transdermal or oral administration, or as an inhalation formulation or suppository. Alternatively, dosage forms may be for parenteral administration, such as intravenous, intrahepatic, or especially subcutaneous, or intramuscular injection forms. Optionally, a pharmaceutically acceptable carrier and/or excipient may be present.

According to one aspect of the invention, a method for manufacture of a medicament for preventing or treating helminth infection, particularly infection by particularly tapeworms (cestodes), flukes (trematodes) and roundworms (nematodes), tapeworm infection, schistosomiasis, ascariasis, dracunculiasis, elephantiasis, enterobiasis, filariasis, hookworm infection, onchocerciasis, trichinosis and/or trichuriasisis provided, comprising the use of a compound according to the above aspect or embodiments of the invention. Medicaments according to the invention are manufactured by methods known in the art, especially by conventional mixing, coating, granulating, dissolving or lyophilizing.

According to one aspect of the invention, a method for preventing or treating helminth infection, particularly the indications mentioned previously, is provided, comprising the administration of a compound according to the above aspects or embodiments of the invention to a patient in need thereof.

The treatment may be for prophylactic or therapeutic purposes. For administration, a compound according to the above aspect of the invention is preferably provided in the form of a pharmaceutical preparation comprising the compound in chemically pure form and optionally a pharmaceutically acceptable carrier and optionally adjuvants. The compound is used in an amount effective against helminth infection. The dosage of the compound depends upon the species, the patient age, weight, and individual condition, the individual pharmacokinetic data, mode of administration, and whether the administration is for prophylactic or therapeutic purposes. The daily dose administered ranges from approximately 1 μg/kg to approximately 1000 mg/kg, preferably from approximately 1 μg to approximately 100 μg, of the active agent according to the invention.

Wherever reference is made herein to an embodiment of the invention, and such embodiment only refers to one feature of the invention, it is intended that such embodiment may be combined with any other embodiment referring to a different feature. For example, any embodiment that defines R′ may be combined with any embodiment that defines T, R² or R³, to characterize a group of compounds of the invention or a single compound of the invention with different properties.

The invention is further characterized, without limitations, by the following examples, from with further features, advantages or embodiments can be derived. The examples are not meant to limit but illustrate the invention.

### SHORT DESCRIPTION OF THE FIGURES

FIG. 1. shows the development of Haemonchus contortus L3 larvae in the presence of test compounds ahpOH and ahpOH1 from experiments performed on four separate days. The number of L3 counted after 7 days of incubation is displayed. Error bars represent ±1 standard deviation.

FIG. 2. shows the development of Haemonchus contortus L3 larvae in the presence of test compounds ahpOH and ahpOH1. Development of L3 is displayed normalized against development in DMSO control wells (100%) from four independent experiments. Non-linear regression was performed (continuous line) to derive LC50 (dotted line). Error bars represent ±1 standard error of the mean (SEM).

### EXAMPLES

### Cytotoxicity and Nematocidal Studies

The toxicity towards a human cervical cancer HeLa and a non-cancerous (MRC-5) cell line was determined using the fluorometric cell viability assay (Resazurin) (Ahmed et al., J. Immunol. Methods 1994, 170, 211.), results of which are shown in Table 1. The compounds were found non toxic towards both cell lines.

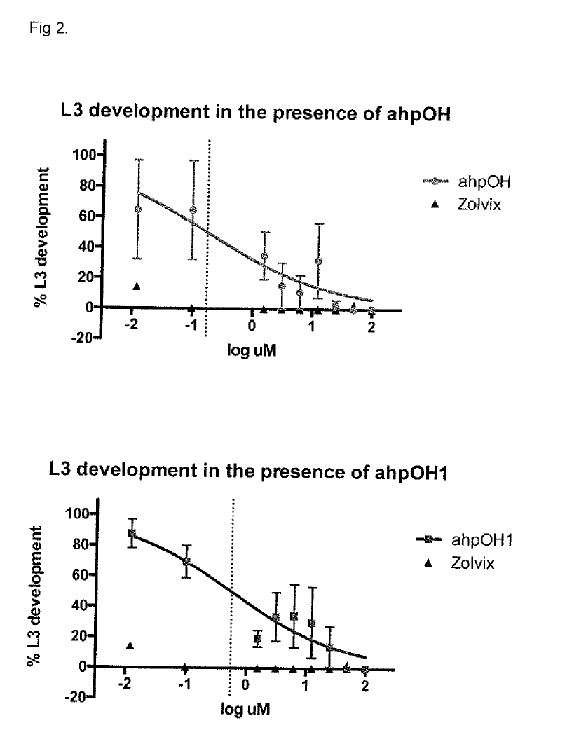

The efficacy of compounds ahpOH and ahpOH1 against H. contortus larval development was assessed and demonstrated using a standard larval development assay (LDA). LDA is an industry-standard test for detecting compounds that have a larvicidal effect against nematodes. LC50 values were 173 nM (95% Cl 19.9 nM 1.495 μM) for compound ahpOH, and 550 nM (95% CI 10.6 nM 2.866 μM) for compound ahpOH1. Dosage-response curves are displayed in Table 1 and FIG. 2.

TABLE 1
Cytotoxicity and nematocidal of selected compounds. IC₅₀ values refer to
measurements against HeLa and MRC-5 cell lines in resazurin assay.

| | IC₅₀ in | IC₅₀ in | LD₅₀/nM against |
|---|---|---|---|
| Compounds | HeLa/μM | MRC-5/μM | H. Contortus |
| 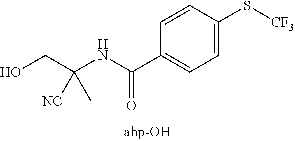 [SMILES: CC(C#N)(CO)NC(=O)c1ccc(SC(F)(F)F)cc1] | >100 | >100 | 173 |
| 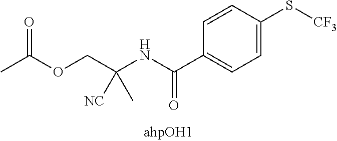 [SMILES: CC(=O)OCC(C)(C#N)NC(=O)c1ccc(SC(F)(F)F)cc1] | >100 | >100 | 550 |
| Zolvix | 34.1 +/− 2.0 | 63.9 +/− 4.3 | <41 |

Compounds ahpOH and ahpOH1 severely and reproducibly inhibited the development of H. contortus larvae at concentrations between 100 μM and 25 μM. Dosage response was demonstrated for both compounds, and LC₅₀s are in the nanomolar range. Efficacy of these compounds was against the parasitic nematode for which the compounds were intended. For both ahpOH and ahpOH1, the concentrations tested in multiple experiments were largely in the micromolar range, and the mean larval development was <50%. Therefore, for both compounds, the area of the curve where LC₅₀s were derived is not highly populated with data points (FIG. 2). There is variation in larval development at concentrations below 25 μM. In these experiments, the confidence in the LC₅₀ calculation is relatively low, as indicated by confidence intervals; further testing of concentrations in the low micromolar to nanomolar range are recommended to achieve an accurate LC₅₀ for each compound.

### Endo Parasites

Activity In Vitro Against Dirofilaria immitis (Di) (Filarial Nematodes).

Freshly harvested and cleaned microfilariae from blood from donor animals (dogs for Di).

The microfilariae are then distributed in formatted microplates containing the test substances to be evaluated for antiparasitic activity. Each compound is tested by serial dilution in order to determine its minimum effective dose (MED). The plates are incubated for 48 hours at 26° C. and 60% relative humidity (RH). Motility of microfilariae is then recorded to identify possible nematocidal activity.

Efficacy is expressed in percent reduced motility as compared to the control and standards.

Activity In Vitro Against Haemonchus contortus & Trichostrongylus colubriformis (Gastro-Intestinal Nematodes).

Freshly harvested and cleaned nematode eggs are used to seed a suitably formatted microplate containing the test substances to be evaluated for antiparasitic activity. Each compound is tested by serial dilution in order to determine its MED. The test compounds are diluted in nutritive medium allowing the full development of eggs through to 3rd instar larvae. The plates are incubated for 6 days at 28° C. and 60% relative humidity (RH). Egg-hatching and ensuing larval development are recorded to identify a possible nematocidal activity.

Efficacy is expressed in percent reduced egg hatch, reduced development of L3, or paralysis & death of larvae of all stages.

The activity against Haemontus Contortus, Dirofilaria immitis and Trychostrongylus colubriformis was tested and the results are shown in table 1a.

TABLE 1a
shows the activity against Haemontus Conturtus, Dirofilaria immitis and
Trychostrongylus colubriformis.

| | Activity against | Activity against | Activity against |
|---|---|---|---|
| | Haemontus | Dirofilaria | Trychostrongylus |
| compounds | Contortus | immitis | colubriformis |
| 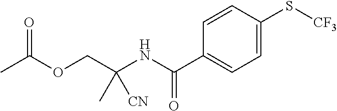 [SMILES: CC(=O)OCC(C)(C#N)NC(=O)c1ccc(SC(F)(F)F)cc1] | EC₉₀ at up to 10 μg/mL | — | EC₇₅ at up to 10 μg/mL |
| 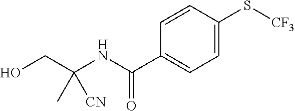 [SMILES: CC(C#N)(CO)NC(=O)c1ccc(SC(F)(F)F)cc1] | EC₅₀ at up to 10 μg/mL | EC₂₀ at up to 10 μg/mL | EC₂₅ at up to 10 μg/mL |
| 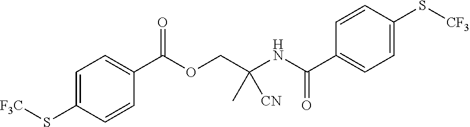 [SMILES: CC(C#N)(COC(=O)c1ccc(SC(F)(F)F)cc1)NC(=O)c1ccc(SC(F)(F)F)cc1] | EC₃₀ at up to 10 μg/mL | — | — |
| 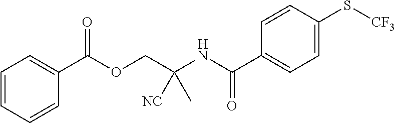 [SMILES: CC(C#N)(COC(=O)c1ccccc1)NC(=O)c1ccc(SC(F)(F)F)cc1] | EC₈₀ at up to 10 μg/mL | EC₆ at up to 10 μg/mL | EC₆₀ at up to 10 μg/mL |
| 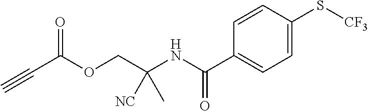 [SMILES: C#CC(=O)OCC(C)(C#N)NC(=O)c1ccc(SC(F)(F)F)cc1] | EC₇₀ at up to 10 μg/mL | EC₁₅ at 0-10 μg/mL | EC₄₀ at up to 10 μg/mL |
| 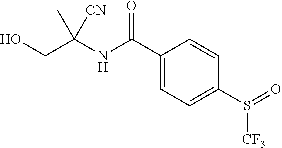 [SMILES: CC(C#N)(CO)NC(=O)c1ccc(S(=O)C(F)(F)F)cc1] | EC₄₅ at up to 10 μg/mL | EC₅ at up to 10 μg/mL | EC₂₅ at up to 10 μg/mL |
| 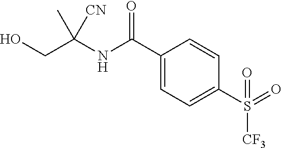 [SMILES: CC(C#N)(CO)NC(=O)c1ccc(S(=O)(=O)C(F)(F)F)cc1] | — | EC₁₅ at up to 10 μg/mL | — |
| 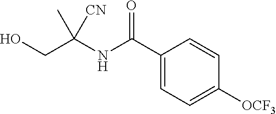 [SMILES: CC(C#N)(CO)NC(=O)c1ccc(OC(F)(F)F)cc1] | EC₆₅ at up to 10 μg/mL | — | EC₅₀ at up to 10 μg/mL |
| 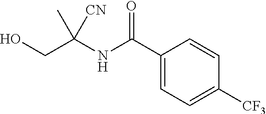 [SMILES: CC(C#N)(CO)NC(=O)c1ccc(C(F)(F)F)cc1] | EC₂₀ at up to 10 μg/mL | EC₂₅ at up to 10 μg/mL | EC₄₀ at up to 10 μg/mL |
| 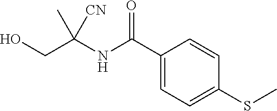 [SMILES: CSc1ccc(C(=O)NC(C)(C#N)CO)cc1] | EC₄₅ at up to 10 μg/mL | EC₁₅ at up to 10 μg/mL | EC₄₀ at up to 10 μg/mL |
| 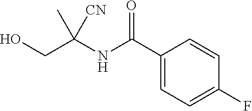 [SMILES: CC(C#N)(CO)NC(=O)c1ccc(F)cc1] | EC₄₀ at up to 10 μg/mL | EC₂₀ at up to 10 μg/mL | EC₃₀ at up to 10 μg/mL |
| 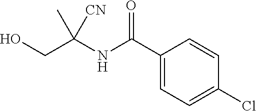 [SMILES: CC(C#N)(CO)NC(=O)c1ccc(Cl)cc1] | EC₅₀ at up to 10 μg/mL | — | EC₄₀ at up to 10 μg/mL |
| 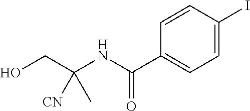 | EC₄₅ at up to 10 μg/ml | EC₁₅ at up to 10 μg/mL | EC₄₅ at up to 10 μg/mL |

As can be seen in Table 1a, interesting EC values could be obtained, especially on Haemontus contortus.

### Experimental Section

### Materials.

All chemicals were of reagent grade quality or better, obtained from commercial suppliers and used without further purification. Solvents were used as received or dried over 4 Å and 3 Å molecular sieves. THF and Et₂O were freshly distilled under N₂ by employing standard procedures.^{[14]} All syntheses were carried out using standard Schlenk techniques.

Instrumentation and methods. ¹H- and ¹³C-NMR spectra were recorded in deuterated solvents on a Bruker DRX 400 or AV2 500 at 30° C. The chemical shiftsδ, are reported in ppm. The residual solvent peaks have been used as internal reference. The abbreviations for the peak multiplicities are as follows: s (singlet), d (doublet), dd (doublet of doublet), t (triplet), q (quartet), m (multiplet) and br (broad). Infrared spectra were recorded on a PerkinElmer spectrum BX TF-IR spectrometer and KBr presslings were used for solids. Signal intensities are abbreviated w (weak), m (medium), s (strong) and br (broad). ESI mass spectra were recorded on a Bruker Esquire 6000 or on a Bruker maxis QTOF-MS instrument (Bruker Daltonics GmbH, Bremen, Germany). High-resolution ESI mass spectra were recorded on a Bruker maxis QTOF-MS instrument (Bruker Daltonics GmbH, Bremen, Germany). The samples (around 0.5 mg) were dissolved in 0.5 mL of MeCN/H₂O 1:1+0.1% HCOOH. The solution was then diluted 10:1 and analysed via continuous flow injection at 3 μl·min⁻¹. The mass spectrometer was operated in the positive electrospray ionization mode at 4000 V capillary voltage, −500 V endplate offset, with a N₂ nebulizer pressure of 0.4 bar and dry gas flow of 4.0 l/min at 180° C. MS acquisitions were performed in the full scan mode in the mass range from m/z 100 to 2000 at 20′000 resolution and 1 scan per second. Masses were calibrated with a 2 mM solution of sodium formate over m/z 158 to 1450 mass range with an accuracy below 2 ppm. Elemental microanalyses were performed on a LecoCJMS-932 elemental analyser.

2-Amino-2-hydroxymethylproprionitrile (ahp)
2-Amino-2-hydroxymethylproprionitrile (ahp) was prepared following the procedure published by Gauvry et al. (WO2005044784A1).
2-Cyano-2-(4-((trifluoromethyl)thio)benzamido)propyl acetate (ahpOH1, 1e)
N-(2-cyano-1-hydroxypropan-2-yl)-4-((trifluoromethyl)thio)benzamide (ahpOH, 0.411 g, 1.35 mmol) was dissolved in dichloromethane (35 mL). To this colorless solution acetyl chloride (144 μL, 2.03 mmol) and NEt₃ (0.28 mL, 0.203 mmol) were added. The reaction mixture was stirred at room temperature for one hour. Then dichloromethane was evaporated and the curde product was purified by column chromatography on silica with hexan:ethyl acetate (7:3) as the eluent (R_{f}=0.59) to give 2-cyano-2-(4-((trifluoromethyl)thio)benzamido)propyl acetate (ahpOH1) as a colorless solid in 44% yield.

Compound 1e, 1d, 1m and 1n were synthesized in a similar fashion as compound ahpOH1 but treated with the appropriate activated carboxylic acid to obtain the desired compound.

Data for ahpOH1

IR (Ker, cm⁻¹): 3478s, 3414s, 2924w, 2845w, 2360w, 2336w, 1757w, 1638m, 1616m, 1533w, 1387w, 1320w, 1225w, 1166w, 1113w, 1079w, 1043w, 856w, 667w, 625w.

¹H NMR (500 MHz, CDCl₃): δ/ppm=7.82 (d, ³J=8.15 Hz, 1H, arom. H), 7.75 (d, ³J=8 Hz, 1H, arom. H), 7.14 (s, 1H, NH), 4.52 (d, ²J=12.0 Hz, 1H, CH₂), 4.40 (d, ²J=11.6 Hz, 1H, CH₂), 2.22 (s, 3H, CH₃), 1.86 (s, 3H, CH₃).

¹³C NMR (500 MHz, CDCl₃): δ/ppm=172.3, 165.8, 136.2, 134.9, 129.5, 129.4, 128.3, 117.8, 67.1, 51.5, 22.2, 21.0.

¹⁹F NMR (400 MHz, CDCl₃): δ/ppm=−41.9.

ESI-MS: m/z (%)=369.05 ([M+Na]⁺, 100), 347.07 ([M+H]⁺, 6).

HR ESI-MS: cald. for C₁₄H₁₄F₃N₂O₃S ([M+H]⁺) m/z (%)=347.06684. found m/z (%)=347.06717.

Anal. Calcd for C₁₄H₁₃F₃N₂O₃S: C, 48.55; H, 3.78; N, 8.09. Found: C, 48.76; H, 3.72; N, 7.95.

### N-(2-cyano-1-hydroxypropan-2-yl)-4-((trifluoromethyl)thio)benzamide (ahpOH, 1a)

After dissolving 2-amino-2-hydroxymethylproprionitrile (ahp, 0.05 g, 0.50 mmol) in dry dichloromethane (5 mL), NEt₃ (70 μl, 0.5 mmol) and 4-(trifluoromethylthio)benzoyl chloride (84 μl, 0.5 mmol) were added and the reaction mixture was stirred for 2 h at room temperature. The solution was extracted with a 1M aqueous solution of hydrochloric acid (2×5 mL). The organic layer was dried over MgSO₄, filtered and the solvent was evaporated under reduced pressure. The residue was suspended in a 1M aqueous solution of NaOH (10 mL) and stirred for 1.5 h at room temperature before THF (10 mL) was added. The solution was stirred for an additional hour. The solvent was evaporated under reduced pressure and the residue was extracted with CH₂Cl₂ (3×10 mL). The combined organic layers were dried over MgSO₄, filtered and the solvent was evaporated under reduced pressure to give N-(2-cyano-1-hydroxypropan-2-yl)-4-((trifluoromethyl)thio)benzamide (ahpOH as a colourless solid. Yield: 32%.

Compounds 1f, 1g, 1h, 1 i, 1j, 1k and 1l were synthesized in the same fashion as compound ahpOH. Compound 1b and 1c could be synthesized in dissolving ahpOH in dry dichloromethane. This colorless reaction solutions were cooled by dry ice to −78° C. To obtain compound 1b, 0.9 equivalents of m-CPBA were added. In case of compound 1c, 2 equivalents of the m-CPBA were needed. After leaving the reaction solutions for 3 h at −78° C., they were then allowed to reach room temperature and the solvent was evaporated. Both compounds were purified using column chromatography on silica.

Data for ahpOH

IR (KBr, cm⁻¹): 3418s, 3288w, 3053w, 2935w, 2845w, 1658m, 1616w, 1591w, 1542m, 1482w, 1456w, 1395w, 1317w, 1135m, 1115m, 1081m, 1012w, 925w, 844w, 763w, 623w.

¹H NMR (500 MHz, CDCl₃): δ/ppm=7.84 (d, ³J=8.0 Hz, 2H, arom. H), 7.75 (d, ³J=8 Hz, 2H, arom. H), 6.63 (s, 1H, NH), 4.06 (d, ²J=10.80 Hz, 1H, CH₂), 3.89 (d, ²J=11.20 Hz, 2H, CH₂), 2.83 (s, 1H, OH), 1.81 (s, 3H, CH₃).

¹³C NMR (500 MHz, MeOD): δ/ppm=167.2, 136.0, 134.9, 129.4, 129.3, 128.5, 119.7, 66.6, 52.8, 21.7.

¹⁹F NMR (500 MHz, CDCl₃): δ/ppm=−39.0.

ESI-MS: m/z (%)=327.04 ([M+Na]⁺, 100), 305.06 ([M+H]⁺, 21).

HR ESI-MS: cald. for C₁₂H₁₂F₃N₂O₂S ([M+H]⁺) m/z (%)=305.05626. found m/z (%)=305.05661.

Anal. Calcd for Cl₂H₁₁F₃N₂O₂S: C, 47.37; H, 3.64; N, 9.21. Found: C, 47.55; H, 3.55; N, 9.02.

### Data for 1d

IR (KBr, cm-1): 3551m, 3467s, 3412s, 3233m, 3047w, 2924w, 2851w, 1733s, 1639s, 1617m, 1534m, 1398w, 1385w, 1362w, 1323w, 1264m, 1166s, 1130s, 1114s, 1079s, 1017w, 878w, 855w, 762w, 688w, 625m, 496w.

¹H NMR (500 MHz, CDCl₃): δ/ppm=8.15 (d, ³J=8.5 Hz, 2H, arom. H), 7.83 (d, ³J=8.5 Hz, 2H, arom. H), 7.78-7.75 (m, 4H, arom. H), 4.78 (dd, ²J=12 Hz, 2H, CH₂), 1.94 (s, 3H, CH₃).

¹³C NMR (500 MHz, CDCl₃): δ/ppm=166.8, 165.7, 136.3, 135.9, 134.8, 131.9, 131.1, 130.4, 129.7, 129.4, 129.3, 117.7, 67.8, 51.8, 22.4.

¹⁹F NMR (300 MHz, CDCl₃): δ/ppm=−38.7, −39.0.

ESI-MS: m/z (%)=531.02 ([M+Na]⁺, 100), 509.04 ([M+H]⁺, 6).

HR ESI-MS: cald. for C₂₀H₁₅F₆N₂O₃S₂ ([M+H]+) m/z (%)=509.04163. found m/z (%)=509.04228.

### Compound 1b:

¹H NMR (500 MHz, MeOD): δ/ppm=8.11 (d, ³J=8.5 Hz, 2H, arom. H), 7.98 (d, ³J=8.0 Hz, 2H, arom. H), 3.92 (dd, ²J=11.5 Hz, ²J=11 Hz, 2H, CH₂), 1.75 (s, 3H, CH₃).

Elemental Analysis: calcd. for C₁₂H₁₁F₃N₂O₃S: C, 45.00; H, 3.46; N, 8.75. Found C, 45.24; H, 3.49; N, 8.61.

### Compound 1c:

¹H NMR (500 MHz, MeOD): δ/ppm=8.21 (d, ³J=9.0 Hz, 2H, arom. H), 8.18 (d, ³J=9.0 Hz, 2H, arom. H), 3.92 (dd, ²J=11.5 Hz, ²J=11 Hz, 2H, CH₂), 1.75 (s, 3H, CH₃).

Elemental Analysis: calcd. for C₁₂H₁₁F₃N₂O₄S: C, 42.86; H, 3.30; N, 8.33. Found C, 42.93; H, 3.35; N, 8.27.

### Compound 1f:

¹H NMR (500 MHz, CDCl₃): δ/ppm=7.81 (d, ³J=11.2 Hz, 2H, arom. H), 7.22 (d, ³J=8.0 Hz, 2H, arom. H), 6.99 (s, 1H, NH), 3.94 (dd, ²J=11.6 Hz, ²J=11.2 Hz, 2H, CH₂), 1.75 (s, 3H, CH₃).

Elemental Analysis: calcd. for C₁₂H₁₁F₃N₂O₃: C, 50.01; H, 3.85; N, 9.72. Found C, 50.27; H, 3.79; N, 9.58.

### Compound 1g:

¹H NMR (500 MHz, CDCl₃): δ/ppm=7.91 (d, ³J=8.5 Hz, 2H, arom. H), 7.73 (d, ³J=8.0 Hz, 2H, arom. H), 6.68 (s, 1H, NH), 4.09-4.05 (m, 1H, CH₂), 3.91-3.88 (m, 1H, CH₂), 1.82 (s, 3H, CH₃).

Elemental Analysis: calcd. for C₁₂H₁₁F₃N₂O₂: C, 52.94; H, 4.07; N, 10.29. Found C, 53.11; H, 4.02; N, 10.32.

### Compound 1h:

¹H NMR (500 MHz, CDCl₃): δ/ppm=7.71-7.25 (m, 4H, arom. H), 6.59 (s, 1H, NH), 3.96 (dd, ²J=11.6 Hz, ²J=11.6 Hz, 2H, CH₂), 2.51 (s, 3H, CH₃), 1.79 (s, 3H, CH₃).

Elemental Analysis: calcd. for C₁₂H₁₄N₂O₂S: C, 57.58; H, 5.64; N, 11.19. Found C, 57.65; H, 5.47; N, 11.05.

### Compound 1i:

¹H NMR (400 MHz, CDCl₃): δ/ppm=7.82-7.80 (m, 2H, arom. H), 7.15-7.12 (m, 2H, arom. H), 6.57 (s, 1H, NH), 4.06-4.03 (m, 1H, CH₂), 3.90-3.86 (m, 1H, CH₂), 1.79 (s, 3H, CH₃).

Elemental Analysis: calcd. for C₁₁H₁₁FN₂O₂: C, 59.45; H, 4.99; N, 12.61. Found C, 59.41; H, 4.96; N, 12.55.

### Compound 1j:

¹H NMR (400 MHz, CDCl₃): δ/ppm=7.75-7.73 (m, 2H, arom. H), 7.45-7.43 (m, 2H, arom. H), 6.54 (s, 1H, NH), 4.47 (dd, ²J=11.2 Hz, ²J=11.2 Hz, 2H, CH₂), 1.80 (s, 3H, CH₃).

Elemental Analysis: calcd. for C₁₁H₁₁ClN₂O₂: C, 55.36; H, 4.65; N, 11.74. Found C, 55.45; H, 4.61; N, 11.40.

### Compound 1k:

¹H NMR (400 MHz, CDCl₃): δ/ppm=7.68-7.60 (m, 4H, arom. H), 6.52 (s, 1H, NH), 3.97 (dd, ²J=11.6 Hz, ²J=11.6 Hz, 2H, CH₂), 1.80 (s, 3H, CH₃).

Elemental Analysis: calcd. for C₁₁H₁₁ClN₂O₂: C, 55.36; H, 4.65; N, 11.74. Found C, 55.45; H, 4.61; N, 11.40.

compound 1l:

¹H NMR (400 MHz, CDCl₃): δ/ppm=7.82 (d, ³J=8.4 Hz, 2H, arom. H), 7.51 (d, ³J=8.8 Hz, 2H, arom. H), 6.51 (s, 1H, NH), 3.96 (dd, ²J=11.6 Hz, ²J=11.2 Hz, 2H, CH₂), 1.80 (s, 3H, CH₃).

Elemental Analysis: calcd. for C₁₁H₁₁IN₂O₂: C, 40.02; H, 3.36; N, 8.49. Found C, 39.74; H, 3.52; N, 8.28.

### Compound 1m:

¹H NMR (400 MHz, CDCl₃): δ/ppm=8.13-8.11 (m, 2H, arom. H), 7.85-7.82 (m, 2H, arom. H), 7.76-7.73 (m, 2H, arom. H), 7.66-7.62 (m, 1H, arom. H), 7.52-7.48 (m, 2H, arom. H), 7.38 (s, 1H, NH), 4.78 (dd, ²J=12 Hz, ²J=11.6 Hz, 2H, CH₂), 1.93 (s, 3H, CH₃).

Elemental Analysis: calcd. for C₁₉H₁₅F₃N₂O₂S: C, 55.88; H, 3.70; N, 6.86. Found C, 55.58; H, 3.62; N, 6.79.

### Compound 1n:

¹H NMR (500 MHz, CDCl₃): δ/ppm=7.81 (d, ³J=8.5 Hz, 2H, arom. H), 7.75 (d, ³J=8.0 Hz, 2H, arom. H), 6.65 (s, 1H, NH), 4.67-4.62 (m, 2H, CH₂), 3.07 (s, 1H, CH), 1.88 (s, 3H, CH₃).

Elemental Analysis: calcd. for C₁₅H₁₁F3N₂O₃S: C, 50.56; H, 3.11; N, 7.86. Found C, 50.59; H, 3.07; N, 7.79.

### Cell Culture.

Human cervical carcinoma cells (HeLa) cells were cultured in DMEM (Gibco) supplemented with 5% fetal calf serum (FCS, Gibco), 100 U/ml penicillin, 100 μg/ml streptomycin at 37° C. and 5% CO₂. The normal human fetal lung fibroblast MRC-5 cell line was maintained in F-10 medium (Gibco) supplemented with 10% FCS (Gibco), penicillin (100 U/ml), and streptomycin (100 μg/ml). Cytotoxicity studies were performed on two different cell lines, namely HeLa, and MRC-5, by a fluorometric cell viability assay using Resazurin (Promocell GmbH). Briefly, one day before treatment, cells were seeded in triplicates in 96-well plates at a density of 4×10³ cells/well for HeLa and 7×10³ for MRC-5 in 1041.1 growth medium. Upon treating cells with increasing concentrations of Fc-PZQ derivatives for 48h, the medium was removed, and 100 μl complete medium containing Resazurin (0.2 mg/ml final concentration) were added. After 4h of incubation at 37° C., fluorescence of the highly red fluorescent product Resorufin was quantified at 590 nm emission with 540 nm excitation wavelength in a SpectraMax M5 microplate Reader.

Haemonchus Infection of Sheep

Helminth-free sheep (six weeks) were purchased, introduced into an indoor animal facility (Parkville, Victoria, Australia), immediately treated with a broad spectrum anthelmintic (including abamectin and a benzimidazole) as well as a coccidiostat (at therapeutic doses) and then allowed to acclimatize for one month. Sheep were then each inoculated via gavage (directly into the reticulo-rumen) with 7,500 to 10,000 third-stage larvae (L3s) of the strain Haecon 5. Sheep were fed with high quality commercial feed (chaff) and provided with water ad libitum. After 25-30 days, infections were patent, and sheep commenced excreting Haemonchus eggs. A standard PCR-based sequencing method (Gasser et al., Nat Protoc. 2006, 1, 3121-8.) was used to demonstrate that the infection was monospecific.

Larval Development Assay (LDA)

### In Vivo Parasite Culture.

The Haecon 5 strain is susceptible to macrocyclic lactones (and reported to be susceptible to aminoacetyl-nitriles, although this has not been tested by the inventors): therefore, the use of monepantel as a positive control in the assay is appropriate. Faeces from these monospecifically infected sheep were collected and used in experiments.

Isolation of H. contortus Eggs from Sheep Faeces.

˜100 g of faeceswerecrushed and suspended in ˜1000 ml of sugar solution (specific gravity 1.2), sieved through a ‘tea strainer’ and undigested plant matter discarded. The sugar solution was then placed into a flat dish and strips of plastic overhead transparency film placed on the surface. The plastic was left for at least 45 minto allow the eggs to stick and then removed carefully. The eggs were collected by washing from the plastic, with water, into a 50 ml centrifuge tube. The water containing eggs was put through a 40 μmsieve to remove further plant material and then centrifuged at 1,000×g for 10 min. Eggs were aspirated, transferred to a fresh tube, washed in 50 ml water (centrifuged as previously), supernatant aspirated and resuspendedin 1 ml of water and then diluted to ˜200 eggs/20 ul.

Dilution and Preparation of Compounds in Solid Agar.

Compounds were stored as powder at −20° C., and diluted in dimethyl sulfoxide (DMSO) to achieve a 100 mM concentration. These stock solutions were diluted further in DMSO to producedilution series at 100× the intended final concentrations; 10 μL of 100× test compound (in 100% DMSO) were added to a 1.5 ml microcentrifuge tube, 1 ml of molten agar added, the tube vortexed and the agar aliquoted (150 μL) into wells of a 96-well microtitre plate (see Table 2).

TABLE 2
Testing of compounds in four independent experiments (using eggs from sheep with
three independent H. Contortus infections). Concentrations were tested in serial dilution,
stepping down by a factor of 2, except where indicated otherwise.

| Compounds | ahpOH | ahpOH1 | ahpOH1 | ahpOH | ahpOH, | ahpOH, |
|---|---|---|---|---|---|---|
| tested | | | | | ahpOH1 | ahpOH1 |
| Concentrations | 100 μM-6.25 μM | | 100 μM-1.9 pM | 100 μM-1.9 pM | 100 μM, 10 μM, | 100 μM-1.56 μM |
| tested (μM) | | | | | 100 nM | |
| | | | | | and 10 nM | |
| No. of replicate | 1 | 1 | 1 | 3 | 2 | 3 |
| wells | | | | | | |
| No. of DMSO- | 16 | 12 | 1 | 1 | 8 | 14 |
| wells averaged | | | | | | |

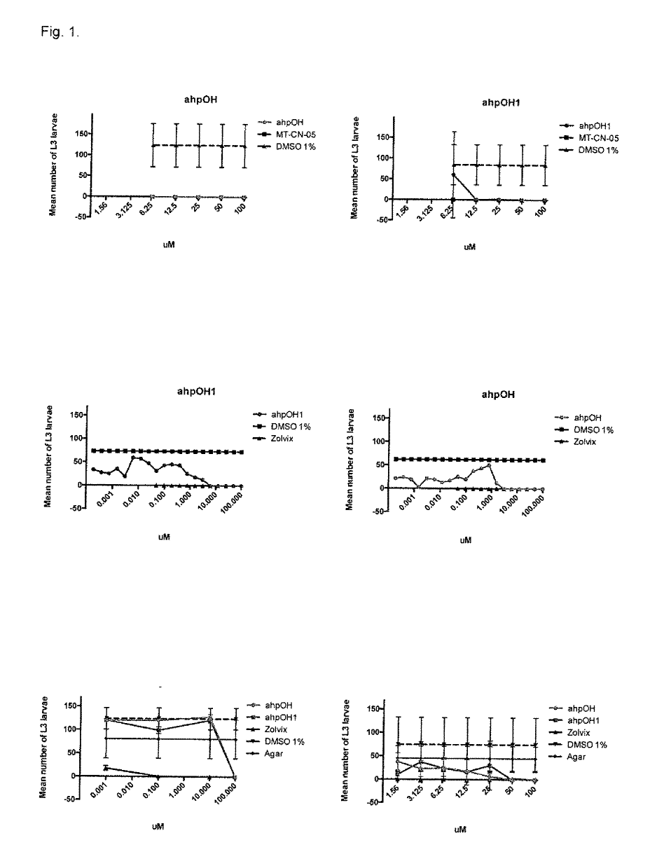

DMSO (1%) was used in a number of wells as a solvent-only control (negative controls), 100% agar was used as a negative control and cydectin was used as a positive control at the same concentrations as test compounds; 200 eggs (20 μL) were added to each well and plates were incubated overnight at 27° C. The following day, plates were checked to ensure that most eggs had hatched in negative control wells. Any compounds that appeared to have an ovicidal effect were noted. 15 μL of nutritive medium was added to feed the larvae. Nutritive medium was prepared as follows: 1 g of yeast extract was added to 90 ml of physiological saline and autoclaved for 20 min at 121° C. Three mL of 10× Earle's Balanced Salt Solution (EBSS) were added to 27 mL of yeast extract solution and the pH of the solution adjusted to 5.4-5.6 through the addition of bicarbonate. Following an additional incubation of 7 days (27° C.), the L3 larvae that had developed in each well were counted (FIG. 1).

Statistical Analysis

For each independent experiment, the number of L3s counted in Zolvix (positive control) and test wells was divided by the mean number of L3 counted in DMSO control wells, and expressed as a percentage of DMSO control development. For simplicity, 10 μM and 12.5 μM test conditions from independent experiments were ‘collapsed’ to a single 12.5 μM data point, and 100 nM and 125 nM data points were collapsed to 125 nM. L3 development data for concentrations that were tested only once were omitted from further analysis. The mean, normalized percentage of L3 for test conditions and Zolvix were charted against the log of concentrations tested, and represented graphically (FIG. 2). Non-linear regression was performed to calculate LC50 values for ahpOH and ahpOH1 using Prism (v.6 GraphPad).

1. A compound characterized by a general formula (A), 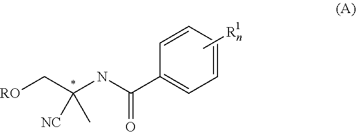
wherein
n of R¹ₙ is 0, 1, 2, 3, 4 or 5 and
each R¹ independently from any other R¹ is —C(═O)OR^{2a}, —C(═O)NR^{2a}₂, —C(═O)SR^{2a}, —C(═S)OR^{2a}, —C(NH)NR^{2a}₂, CN₄H₂, —NR^{2a}₂, —C(═O)R^{2a}, —C(═S)R^{2a}, —OR^{2a}, —SR^{2a}, —CF₃, —OCF₃, —SCF₃, —SOCF₃, —SO₂CF₃, —CN, —NO₂, —F, —Cl, —Br or —I,
with each R^{2a} independently from any other R^{2a} being a hydrogen or C₁-C₄ alkyl, and
R is hydrogen or CO—R′
wherein R is
an unsubstituted or substituted C₁-C₁₀ alkyl, an unsubstituted or substituted C₂-C₁₀ alkenyl, an unsubstituted or substituted C₂-C₁₀ all an unsubstituted or substituted C₃-C₈ cycloalkyl, an unsubstituted or substituted alkoxy, an unsubstituted or substituted C₃-C₈ cycloalkoxy,
an unsubstituted or substituted C₆-C₁₄, aryl,
an unsubstituted or substituted 5- to 10-membered heteroaryl, wherein 1 to 4 ring atoms are independently selected from nitrogen, oxygen or sulfur,
an unsubstituted or substituted 5- to 10-membered heteroalicyclic ring, wherein 1 to 3 ring atoms are independently nitrogen, oxygen or sulfur,
—OR², —C(O)R², —C(O)OR², —C(O)NR²R³, —NR²R³, —S(O)₂R², —S(O)₂OR², and —S(O)₂NR²R³,
wherein
R² and R³ are independently selected from the group consisting of hydrogen, unsubstituted C₁-C₄ alkyl, and C₁-C₄ alkyl substituted with C₁-C₄ alkoxy;
for use in a method for treatment of infections by helminths,
or for use in a method to suppress plant helminths.

2. The compound according to claim 1 characterized by a general formula (1), 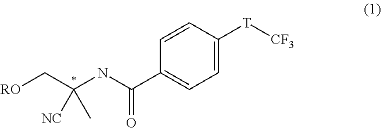
wherein T is S, SO or SO₂, and
R is hydrogen or CO—R′
wherein R′ is
an unsubstituted or substituted C₁-C₁₀ alkyl, an unsubstituted or substituted C₂-C₁₀ alkenyl, an unsubstituted or substituted C₂-C₁₀ alkynyl, an unsubstituted or substituted C₃-C₈ cycloalkyl, an unsubstituted or substituted C₁-C₁₀ alkoxy, an unsubstituted or substituted C₁-C₈ cycloalkoxy,
an unsubstituted or substituted C₆-C₁₄ aryl,
an unsubstituted or substituted 5- to 10-membered heteroaryl, wherein 1 to 4 ring atoms are independently selected from nitrogen, oxygen or sulfur,
an unsubstituted or substituted 5- to 10-membered heteroalicyclic ring, wherein 1 to 3 ring atoms are independently nitrogen, oxygen or sulfur,
—OR², —C(O)R¹, —C(O)OR², —C(O)NR²R³, —NR²R³, —S(O)₂R², —S(O)₂OR², and —S(O)₂NR²R³,
wherein
R² and R³ are independently selected from the group consisting of hydrogen, unsubstituted C₁-C₄ alkyl, and C₁-C₄ alkyl substituted with C₁-C₄ alkoxy;
for use in a method for treatment of infections by helminths,
or for use in a method to suppress plant helminths.

3. The compound according to claim 1, wherein R′ is selected from
C₁-C₄ alkyl
an unsubstituted or substituted C₆-C₁₄ aryl,
an unsubstituted or substituted 5- to 10-membered heteroaryl, wherein 1 to 4 ring atoms are independently selected from nitrogen, oxygen or sulfur.

4. The compound according to claim 3, wherein R′ is methyl or an aryl or a heteroaryl selected from the group comprised of: 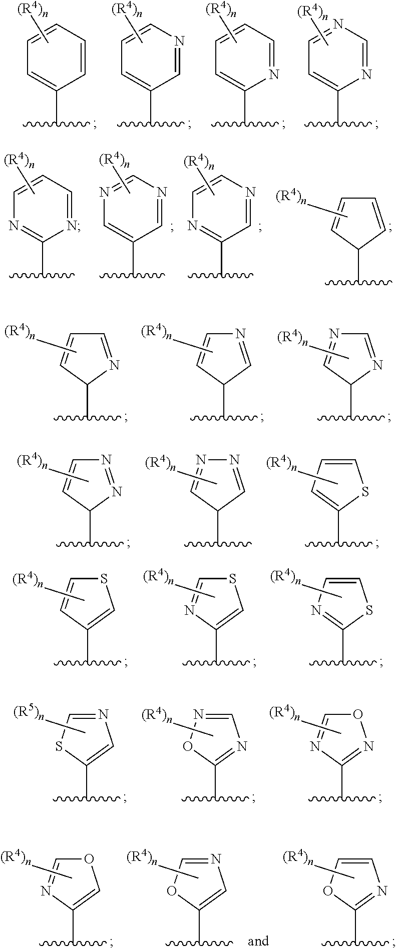
wherein
n is 0, 1, 2, 3 or 4, and
each R⁴ independently from any other is COOR⁵, CONR⁵₂, C(NH)NR⁵₂, CN₄H₂, NR⁵₂, COR⁵, OR⁵, CF₃, OCF₃, SCF₃, SOCF₃, SO₃CF₃, CN, NO₂, F, Cl or Br,
with each R⁵ independently from any other being hydrogen or a C₁-C₄ alkyl.

5. The compound according to claim 4, wherein R′ is selected from the group comprised of: 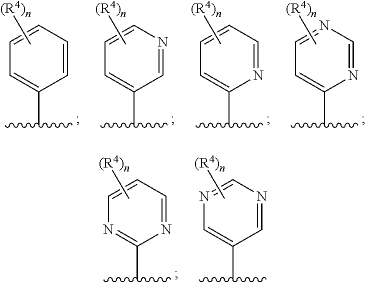
wherein n is 1 and R⁴ is CF₃, OCF₃, SCF₃, SOCF₃, or SO₂CF₃.

6. The compound according to claim 1, wherein R′ is selected from
methyl,
4-(trifluoromethylsufanyl)phenyl,
4-(trifluromethylsulfinyl)phenyl, or
4-(trifluoromethylsulfonyl)phenyl.

7. The compound according to claim 1 for use in a method for treatment of infection by helminths selected from
a. N-(1-cyano-2-hydroxy-1-methyl-ethyl)-4-(trifluoromethylsulfanyl)benzamide;
b. N-(1-cyano-2-hydroxy-1-methyl-ethyl)-4-(trifluoromethylsulfinyl)benzamide
c. N-(1-cyano-2 hydroxy-1-methyl-ethyl)-4-(trifluoromethylsulfonyl)benzamide
d. [2-cyano-2-[[4-(trifluoromethylsulfanyl)benzoyl]amino]propyl]4-(trifluoromethylsulfanyl)benzoate
e. [2-cyano-2-[[4-(trifluoromethylsulfanyl)benzoyl]amino]propyl]acetate
f. N-(2-cyano-1-hydroxypropan-2-yl)-4-(trifluoromethoxy)benzamide
g. N-(2-cyano-1-hydroxypropan-2-yl)-4-(trifluoromethyl)benzamide
h. N-(2-cyano-1-hydroxypropan-2-yl)-4-(methylthio)benzamide
i. N-(2-cyano-1-hydroxypropan-2-yl)-4-fluorobenzamide
j. 4-chloro-N-(2-cyano-1-hydroxypropan-2-yl)benzamide
k. 4-bromo-N-(2-cyano-1-hydroxypropan-2-yl)benzamide
l. N-(2-cyano-1-hydroxypropan-2-yl)-1-iodobenzamide
m. 2-cyano-2-(4-((trifluoromethyl)thio)benzamido)propyl benzoate
n. 2-cyano-2-(4-((trifluoromethyl)thio)benzamido)propyl propiolate

8. A compound characterized by a general formula (B), 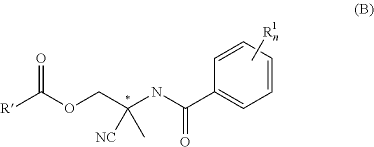
wherein
n of R¹ₙ is 0, 1, 2, 3, 4 or 5, and
each R¹ independently from any other R¹ is —C(═O)OR^{2a}, —C(═O)NR^{2a}₂, —C(═O)SR^{2a}, —C(═)OR^{2a}, —C(NH)NR^{2a}₂, CN₄H₂, —NR^{2a}₂, —C(═O)R^{2a}, —C(═S)R^{2a}, —OR^{2a}, —SR^{2a}, —CF₃, —OCF₃, —SCF₃, —SOCF₃, —SO₂CF₃, —CN, —NO₂, —F, —Cl, —Br or —I,
with each R^{2a} independently from any other R^{2a} being a hydrogen or C₁-C₄ alkyl, and
R′ is
an unsubstituted or substituted C₁-C₁₀ alkyl, an unsubstituted or substituted C₂-C₁₀ alkenyl, an unsubstituted or substituted C₂-C₁₀ alkynyl, an unsubstituted or substituted C₃-C₈ cycloalkyl, an unsubstituted or substituted C₁-C₁₀ alkoxy, unsubstituted or substituted C₃-C₈ cycloalkoxy,
an unsubstituted or substituted C₆-C₁₄ aryl,
an unsubstituted or substituted 5- to 10-membered heteroaryl, wherein 1 to 4 ring atoms are independently selected from nitrogen, oxygen or sulfur,
unsubstituted or substituted 5- to 10-membered heteroalicyclic ring, wherein 1 to 3 ring atoms are independently nitrogen, oxygen or sulfur,
—OR², —C(O)R¹, —C(O)OR², —C(O)NR²R³, —NR²R³, —S(O)₂R², —S(O)₂OR², and —S(O)₂NR²R³,
wherein
R² and R³ are independently selected from the group consisting of hydrogen, unsubstituted C₁-C₄ alkyl, and C₁-C₄ alkyl substituted with C₁-C₄ alkoxy.

9. The compound according to claim 8 characterized by a general formula (2), 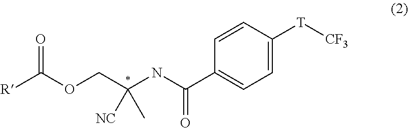
wherein T is 5, SO or SO₂, and
R′ is
an unsubstituted or substituted alkyl, an unsubstituted or substituted C₂-C₁₀ alkenyl, an unsubstituted or substituted C₂-C₁₀ alkynyl, an unsubstituted or substituted C₃-C₈ cycloalkyl, an unsubstituted or substituted C₁-C₁₀ alkoxy, an unsubstituted or substituted C₃-C₈ cycloalkoxy,
an unsubstituted or substituted C₆-C₁₄ aryl,
an unsubstituted or substituted 5- to 10-membered heteroaryl, wherein 1 to 4 ring atoms are independently selected from nitrogen, oxygen or sulfur,
an unsubstituted or substituted 5- to 10-membered heteroalicyclic ring, wherein 1 to 3 ring atoms are independently nitrogen, oxygen or sulfur,
—OR², —C(O)R¹, —C(O)OR², —C(O)NR²R³, —NR²R³, —S(O)₂R², —S(O)₂OR², and —S(O)₂NR²R³,
wherein
R² and R³ are independently selected from the group consisting of hydrogen, unsubstituted C₁-C₄ alkyl, and C₁-C₄ alkyl substituted with C₁-C₄ alkoxy.

10. The compound according to claim 8, wherein R′ is selected from
an unsubstituted or substituted C₆-C₁₄ aryl,
unsubstituted or substituted 5- to 10-membered heteroaryl, wherein 1 to 4 ring atoms are independently selected from nitrogen, oxygen or sulfur.

11. The compound according to claim 10, wherein R′ is methyl or an aryl or a heteroaryl selected from the group comprised of: 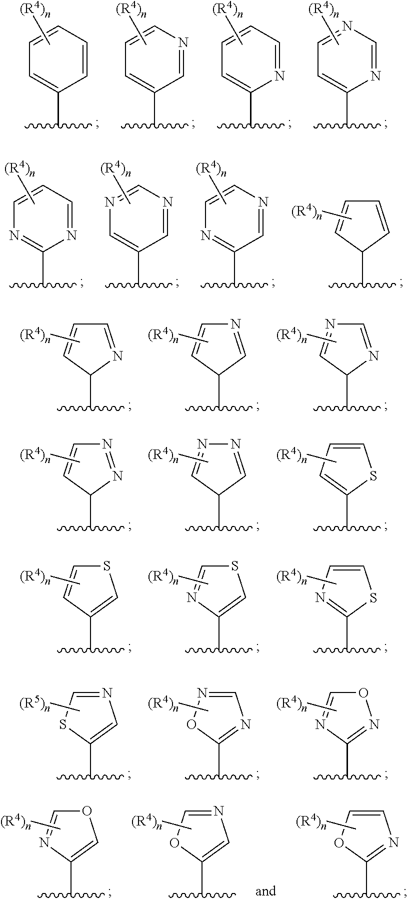
wherein
n is 0, 1, 2, 3 or 4, and
each R⁴ independently from any other is COOR⁵, CONR⁵₂, C(NH)NR⁵₂, CN₄H₂, NR⁵₂, COR⁵, OR⁵, CF₃, OCF₃, SCF₃, SOCF₃, SO₂CF₃, CN, NO₂, F, Cl or Br,
with each R⁵ independently from any other being hydrogen or a C₁-C₄ alkyl.

12. The compound according to claim 11, wherein R′ is selected from the group comprised of: 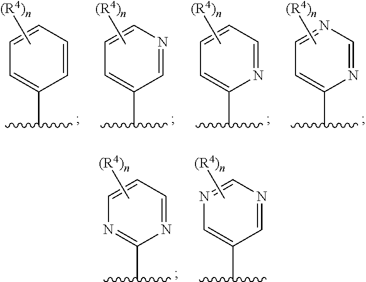
wherein n is 1 and R⁴ is CF₃, OCF₃, SCF₃, SOCF₃, or SO₂CF₃.

13. The compound according to claim 8, wherein R′ is selected from
methyl,
4-(trifluoromethylsulfanyl)phenyl,
4-(trifluoromethylsulfinyl)phenyl, or
4-(trifluoromethylsulfonyl)phenyl.

14. A compound according to claim 1, wherein the C1 carbon atom of the ethyl moiety is in the S configuration.

15. A compound according to claim 8, wherein the C1 carbon atom of the ethyl moiety is in the S configuration.

16. A compound according to claim 8, for use in a method of treatment of disease, wherein in particular the C1 carbon atom of the ethyl moiety is in the S configuration.

17. A compound according to claim 14 for use in a method of treatment of disease, wherein in particular the C1 carbon atom of the ethyl moiety is in the S configuration.
